(19) United States
(12) Patent Application Publication
(10) Pub. No.: US 2008/0234310 A1
(43) Pub. Date: Sep. 25, 2008

(54) Methods and Compositions for Slowing Aging

(76) Inventors: Sergei O. Bachurin, Chernogolovka (RU); Vladimir V. Grigoriev, Chernogolovka (RU)

Correspondence Address: MORRISON & FOERSTER LLP, 755 PAGE MILL RD, PALO ALTO, CA, 94304-1018, US

(21) Appl. No.: 10/582,312

(22) PCT Filed: Dec. 8, 2004
(86) PCT No.: PCT/US2004/041081

§ 371 Date: Mar. 11, 2008

(30) Foreign Application Priority Data
Dec. 8, 2003 (RU) 2003135482

(51) Int. Cl. A61K 31/437; A61P 43/00

(52) U.S. Cl. 514/292

(57) ABSTRACT

The present invention provides a pharmacological agent based on derivatives of hydrogenated pyrido (4,3-b) indoles and uses thereof. Specifically, the invention provides a geroprotector in the series of hydrogenated pyrido (4,3-b) indoles (several derivatives), which can be used for slowing aging, prolonging lifespan of an individual or cells in an individual, and/or improving quality of life of an individual developing or having a risk of developing age-associated manifestations and/or pathologies.

### CROSS-REFERENCE TO RELATED APPLICATIONS

This application claims priority to Russian Patent Application No. 2003135482, registered on Dec. 8, 2003, which is incorporated herein by reference in its entirety.

### STATEMENT REGARDING FEDERALLY SPONSORED RESEARCH OR DEVELOPMENT

Not applicable.

### TECHNICAL FIELD

The present invention is related to the use of hydrogenated pyrido (4,3-b) indoles or pharmaceutically acceptable salt thereof in the area of medicine, which may be used as geroprotective agents when they are prepared as pharmacological compositions for slowing aging, and/or for the prolongation of life and/or for the improvement of the quality of life.

### BACKGROUND OF THE INVENTION

Old age is characterized by a significant increase of the probability of death. In addition, it is characterized by a sharp increase in a probability of occurrence of various pathologies and conditions that are not life threatening, but are associated with the aging process. Such pathologies and conditions in mammals include, for example, loss of sight (cataract), deterioration of the dermatohairy integument (alopecia), and an age-associated decrease in weight due to the death of muscular and fatty cells.

During the recent years, there is a significant interest in the world in identifying new medications and agents, which are aimed to the solution of this problem.

Vitamins A, C, and E increased the duration of life in the experiment disclosed by Baker, G. T. (Baker, G. T., Effects of various antioxidants on ageing in Drosophila//Toxicol Ind. Health.—1993—Vol. 9—p. 163-186). However, hyper saturation of the organism with these vitamins may result in a quick development of hypervitominosis, and may have a negative effect on the functional state of body systems and organs.

There are known agents, which express geroprotective and antioxidant activity, based on the compound ethoxyquin (santoquin). This compound increased the duration of life of C3H mice, when it was added to the food (Comfort, A., Youhotsky-Gore, J., Pathmanatchan, K., Effect of ethoxyquin on the longevity of C3H mice//Nature.—1971—Vol. 229—p. 254-255). Longevity of life of the laboratory animals was also increased as a result of the administration of a low toxic water soluble antioxidant 2-ethyl-6-methyl-3-hydroxypyridine chlorohydrate, which is a structural analog of the vitamin B6 (Obukhova, L. K., Chemical geroprotectors, prolongation of life//Uspekhi Khimii (Russ.)—1975, Vol. 44—p. 1914-1925). Insignificant prolongation of life was observed in experiments with 2-mercaptoethanol amine, cysteine, centrophenoxine, butylhydroxyl toluene, glutathione, 3-hydroxypyridine, lactic acid, and gluconic acid (Frolkis, V. V., Muradyan, H. K., Experimental methods of the prolongation of life (Russ.)//Leningrad, Nauka, 1988; Obukhova, L. K., Emmanuel, N. M., Molecular mechanisms of the delay of ageing with antioxidants//Obschie problemy biologii (Russ.)/VINITI.—Vol. 4—p. 44-80).

However, these chemical compounds did not find their application as therapeutic agents in medicine.

A therapeutic agent gerovital, which contains procaine, is used as a geroprotective medication (Mashkovsky, M. D., Medicinal drugs (Russ.).—Moscow, Medicina, 1993—part 1—chapter 3—p. 375). However, there were incidents of its negative effect on the cardio-vascular functions, disturbances in sleep, anxiety, and muscle aches and joints aches.

Compounds with geroprotective properties, which were discovered during the recent years, are endogenous compounds melatonin and N-acetylserotonin (NAS). These compounds have antioxidant properties and, according to one of theories on the mechanism of ageing, should have geroprotective effect (Heng-Long Hu, Forsey, R. J., Blades, T. J., Barrat, M. E. J., Parmar, P., Powell, J. R. Antioxidants may contribute in the fight against ageing: an in vitro model. Mechanisms of Ageing and Development 121 (2000) 217-230). Indeed, experiments with C57B1 mice demonstrated that melatonin and its precursor NAS were capable of prolonging the life of the male mice, when they received these compounds from the age of 2 months. However, these compounds were ineffective in experiments with male mice of the same line, when animals received these compounds from the age of 12 months (Oxenkrug, G., Requintina, P., Bachurin, S. Antioxidant and Anti-Ageing Activity of N-acetylserotonin and Melatonin in the in vivo models. Ann. N.Y. Acad. Sci. 2001, v. 939, p. 190-199).

The problem, to which solution the present invention was targeted, was to expand the range of therapeutic tools, which may be used as new effective geroprotectors that prolong life and improve the quality of life.

All references, publications, patents, and patent applications disclosed herein are hereby incorporated by reference in their entirety.

### BRIEF SUMMARY OF THE INVENTION

The present invention provides methods and compositions for slowing aging and/or for improving quality of life and/or for prolongation of life comprising administering to an individual an effective amount of a hydrogenated pyrido (4,3-b) indole or pharmaceutically acceptable salt thereof. The hydrogenated pyrido (4,3-b) indole can be a tetrahydro pyrido (4,3-b) indole or pharmaceutically acceptable salt thereof. The hydrogenated pyrido (4,3-b) indole can be a hexahydro pyrido (4,3-b) indole or pharmaceutically acceptable salt thereof. In one aspect, the invention provides a method of prolonging the lifespan of an individual. In another aspect, the invention provides a method of prolonging the lifespan of cells in an individual, such as cells that respond to calcium influx, including cardiac cells, neurons, glial cells and the like. The cells may be normal cells. The cells may be uninjured cells. In another aspect, the invention provides a method of slowing aging in an individual, for example by delaying the onset and/or slowing the progression of an aging-associated or age-related manifestation and/or pathology or condition, including, but not limited to, disturbance in skin-hair integument (such as baldness or alopecia), vision disturbance (such as development of cataracts), and weight loss (including weight loss due to the death of muscular and/or fatty cells). In another aspect, the invention provides a method of improving the quality of life of an individual, such as an individual developing or at risk of developing these aging-associated or age-related manifestations and/or pathologies. The aging-associated pathologies or conditions are not life-threatening. The invention provides a method of decreasing the risk of developing an age-related pathology or condition.

In one aspect, a method of slowing aging in a mammal is provided, the method comprising administering to a mammal an amount of a hydrogenated pyrido (4,3-b) indole or pharmaceutically acceptable salt thereof effective to slow aging.

In another aspect, a method of slowing the progression of age associated hair loss in a mammal is provided, the method comprising administering to a mammal an amount of a hydrogenated pyrido (4,3-b) indole or pharmaceutically acceptable salt thereof effective to slow the progression of age associated hair loss.

In another aspect, a method of slowing the progression of age associated weight loss in a mammal is provided, the method comprising administering to a mammal an amount of a hydrogenated pyrido (4,3-b) indole or pharmaceutically acceptable salt thereof effective to slow the progression of age associated weight loss.

In another aspect, a method of slowing the onset of an age associated vision disturbance in a mammal is provided, the method comprising administering to a mammal an amount of a hydrogenated pyrido (4,3-b) indole or pharmaceutically acceptable salt thereof effective to slow the onset of an age associated vision disturbance.

In another aspect, a method of improving the quality of life of a mammal is provided, the method comprising administering to a mammal an amount of a hydrogenated pyrido (4,3-b) indole or pharmaceutically acceptable salt thereof effective to improve the quality of life of the mammal.

In another aspect, a method for improving the quality of life of a mammal for which slowing aging is desired is provided, the method comprising administering to a mammal for which slowing aging is desired an amount of a hydrogenated pyrido (4,3-b) indole or pharmaceutically acceptable salt thereof effective to enhance the quality of life of the mammal.

In another aspect, a method for improving the quality of life of a human who desires to slow aging is provided, the method comprising administering to a human who desires to slow aging an amount of a hydrogenated pyrido (4,3-b) indole or pharmaceutically acceptable salt thereof effective to enhance the quality of life of the mammal.

In another aspect, a method of prolonging the lifespan of a mammal is provided, the method comprising administering to a mammal an amount of a hydrogenated pyrido (4,3-b) indole or pharmaceutically acceptable salt thereof effective to prolong the lifespan of the mammal.

In another aspect, a method of extending the lifespan of a cell in a mammal is provided, the method comprising administering to a mammal an amount of a hydrogenated pyrido (4,3-b) indole or pharmaceutically acceptable salt thereof effective to extending the lifespan of a cell in the mammal.

Any of the methods described can use any hydrogenated pyrido (4,3-b) indole or pharmaceutically acceptable salt thereof described throughout this application. For instance, any of the methods described can employ a tetrahydro pyrido (4,3-b) indole or pharmaceutically acceptable salt thereof. Any of the methods described can employ a hexahydro pyrido (4,3-b) indole or pharmaceutically acceptable salt thereof. Any of the methods described can use a hydrogenated pyrido (4,3-b) indole of the Formula A or B:

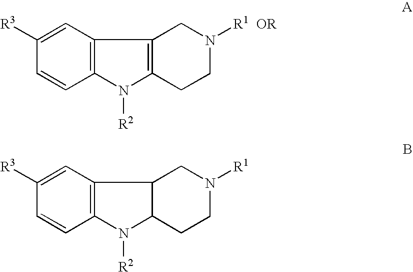

wherein: R¹ is selected from a lower alkyl or aralkyl; R² is selected from a hydrogen, aralkyl or substituted heteroaralkyl and R³ is selected from hydrogen, lower alkyl or halo or any pharmaceutically acceptable salt thereof. Any of the methods described can use a hydrogenated pyrido (4,3-b) indole of the formula A or B, where R¹ is selected from a lower alkyl or PhCH₂—; R² is selected from a hydrogen, PhCH₂— or 6-CH₃-3-Py-(CH₂)₂— and R³ is selected from hydrogen, lower alkyl or halo, or any pharmaceutically acceptable salt thereof. Any of the methods described can use a hydrogenated pyrido (4,3-b) indole of the formula A or B, where R¹ is selected from CH₃—, CH₃CH₂—, or PhCH₂—; R² is selected from H—, PhCH₂—, or 6-CH₃-3-Py-(CH₂)₂—; and R³ is selected from H—, CH₃— or Br—, or any pharmaceutically acceptable salt thereof. Any of the methods described can use a hydrogenated pyrido (4,3-b) indole selected from the group consisting of: cis(±) 2,8-dimethyl-2,3,4,4a,5,9b-hexahydro-1H-pyrido[4,3-b]indole as a racemic mixture or in the substantially pure (+) or substantially pure (−) form; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; or 2-methyl-8-bromo-2,3,4,5-tetrahydro-1H-pyrido[4,3-b]indole or any pharmaceutically acceptable salt of any of the foregoing. Any of the methods described can use 2,8-dimethyl-5-(2-(6-methyl-3-pyridyl)ethyl-2,3,4,5-tetrahydro-1H-pyrido[4,3-b]indole (dimebon) or any pharmaceutically acceptable salt thereof, such as an acid salt, a hydrochloride salt or a dihydrochloride salt thereof. Any of the methods described can use a pharmaceutically acceptable acid salt of any of the hydrogenated pyrido (4,3-b) indoles described herein. Any of the methods described can use a hydrogenated pyrido (4,3-b) indole of the Formula A or B where R¹ is CH₃—, R² is H and R³ is CH₃— or any pharmaceutically acceptable salt thereof. Any of the methods described can use a hydrogenated pyrido (4,3-b) indole of the Formula A or B where R¹ CH₃CH₂— or PhCH₂—, R² is H—, and R³ is CH₃— or any pharmaceutically acceptable salt thereof. Any of the methods described can use a hydrogenated pyrido (4,3-b) indole of the formula A or B where R¹ is CH₃—, R² is PhCH₂—, and R³ is CH₃— or any pharmaceutically acceptable salt thereof. Any of the methods described can use a hydrogenated pyrido (4,3-b) indole of the formula A or B where R¹ is CH₃—, R² is 6-CH₃-3-Py-(CH₂)₂—, and R³ is H— or any pharmaceutically acceptable salt thereof. Any of the methods described can use a hydrogenated pyrido (4,3-b) indole of the formula A or B where R² is 6-CH₃-3-Py-(CH₂)₂— or any pharmaceutically acceptable salt thereof. Any of the methods described can use a hydrogenated pyrido (4,3-b) indole of the formula A or B where R¹ is CH₃—, R² is H—, and R³ is H— or CH₃— or any pharmaceutically acceptable salt thereof. Any of the methods described can use a hydrogenated pyrido (4,3-b) indole of the formula A or B where R¹ is CH₃—, R² is H—, and R³ is Br— or any pharmaceutically acceptable salt thereof.

The hydrogenated pyrido (4,3-b) indole compounds can be tetrahydro pyrido (4,3-b) indole compounds or hexahydro pyrido (4,3-b) indole compounds. The hydrogenated pyrido (4,3-b) indole compounds can be substituted with 1 to 3 substituents, although unsubstituted hydrogenated pyrido (4,3-b) indole compounds or hydrogenated pyrido (4,3-b) indole compounds with more than 3 substituents are also contemplated. The hydrogenated pyrido (4,3-b) indole compounds can be of the formula:

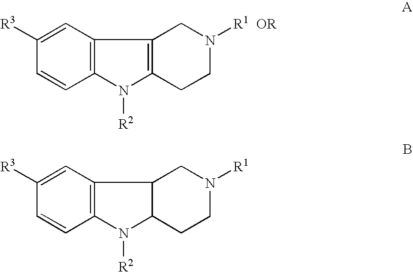

where R¹ is selected from a lower alkyl or aralkyl, R² is selected from a hydrogen, aralkyl or substituted heteroaralkyl and R³ is selected from hydrogen, lower alkyl or halo. Any combination of the substituents is contemplated. Particular compounds for use in the methods disclosed herein include: cis(±) 2,8-dimethyl-2,3,4,4a,5,9b-hexahydro-1H-pyrido[4,3-b]indole as a racemic mixture or the individual compounds in the (+) or (−) forms; 2-ethyl-2,3,4,5-tetrahydro-1H-pyrido[4,3-b]indole; 2-benzyl-2,3,4,5-tetrahydro-1H-pyrido[4,3-b]indole; 2,8-dimethyl-5-benzyl-2,3,4,5-tetrahydro-1H-pyrido[4,3-b]indole; 2-methyl-5-(2-methyl-3-pyridyl)ethyl-2,3,4,5-tetrahydro-1H-pyrido[4,3-b]indole; 2,8-dimethyl-5-(2-(6-methyl-3-pyridyl)ethyl)-2,3,4,5-tetrahydro-1H-pyrido[4,3-b]indole; 2-methyl-2,3,4,5-tetrahydro-1H-pyrido[4,3-b]indole; 2,8-dimethyl-2,3,4,5-tetrahydro-1H-pyrido[4,3-b]indole; 2-methyl-8-bromo-2,3,4,5-tetrahydro-1H-pyrido[4,3-b]indole and any pharmaceutically acceptable salts or forms thereof, including pharmaceutically acceptable acid salts, such as hydrochloric acid salts or dihydrochloric acid salts.

Any of the compounds of Formula B or 1 described herein can be either in the cis or trans form. Any of the compounds of Formula B or 1 described herein can also be present as a racemic mixture (±), as substantially pure compounds (+) or (−), or as any non-racemic mixture. The cis (±) variation, the cis (+) variation and the cis (−) are considered, as well as the trans (±) variation, the trans (+) variation and the trans (−) variation or any combination thereof.

In some embodiments, the compound is administered to an individual who manifests one or more signs of aging, including, but not limited to, hair loss (including baldness), wrinkles, grey hair, and weight loss (including weight loss due to the death of muscular and fatty cells). Age-associated pathologies and conditions are more likely to be present in an older mammal, such as when a mammal is from about middle-age into old age, and the methods and uses can be used for such mammals. In some embodiments, the compound is administered to an individual who is elderly. In some embodiments, the compound is administered to a human who is at least about 35 years old and/or less than about 60 years old. In some embodiments, the compound is administered to a human who is at least about 40, about 45, about 50, about 55, about 60, about 65, about 70, about 75, about 80, about 85, about 90, about 95, about 100, about 105, about 110, about 115 and about 120 years old. In some embodiments, the compound is administered to a human who has not been diagnosed with a neurological disease (such as Alzheimer's disease). In some embodiments, the compound is administered to an individual (such as a human) who has not been diagnosed with cognition impairment associated with aging. In some embodiments, the compound is administered to an individual (such as a human) who does not display a symptom of cognitive impairment. In some embodiments, the compound is administered to an individual which may be any of: bovine, primate, equine, canine, feline, porcine, and ovine animals.

The compound can be administered to an individual continuously (for example, at least once daily) over a sustained period. In some embodiments, the compound is administered to an individual for at least about three months. In some embodiments, the compound is administered to an individual for at least about six months. In some embodiments, the compound is administered to an individual for at least about twelve months.

Use of the hydrogenated pyrido (4,3-b) indoles of formula (1) as medications for the prophylactics of untimely ageing—geroprotectors (anti-ageing medication) for mammals is considered

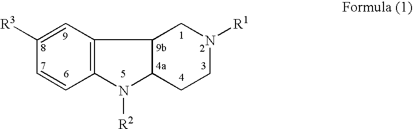

where
R¹ is CH₃—, CH₃CH₂—, or PhCH₂—;
R² is H—, PhCH₂—, or 6-CH₃-3-Py-(CH₂)₂—,
R³ is H—, CH₃—, or Br—;

Also included is the use of compounds of Formula (1), where R¹ is CH₃—, R² is H—, and R³ is CH₃—, where the substance is in either cis or trans isomer form. Also included is the use of compounds of Formula (1) where the compounds are in the form of salts with any pharmaceutically applicable acids or in a form of quaternized derivatives. In one use, the mammal is a human. In one use, any of the described compounds or compositions is used for the prevention of cataract. In one use, any of the described compounds or compositions is used for the prevention of alopecia.

Use of the hydrogenated pyrido (4,3-b) indoles of Formula (2) as medications for the prophylactics of untimely ageing—geroprotectors (anti-ageing medication) for the mammals is considered

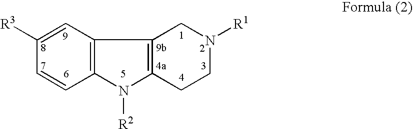

where
R¹ is CH₃—, CH₃CH₂—, or PhCH₂—;
R² is H—, PhCH₂—, or 6-CH₃-3-Py-(CH₂)₂—;
R³ is H—, CH₃—, or Br—;

Also included is the use of compounds of Formula (2), where R¹ is CH₃CH₂— or PhCH₂—, R² is H—, and R³ is CH₃—. Also included is the use of compounds of Formula (2), where R¹ is CH₃—, R² is PhCH₂—, and R³ is CH₃—. Also included is the use of compounds of Formula (2), where R¹ is CH₃—, R² is 6-CH₃-3-Py-(CH₂)₂—, and R³ is H—. Also included is the use of compounds of formula (2), where R¹ is CH₃—, R² is 6-CH₃-3-Py-(CH₂)₂—, and R³ is CH₃—. Also included is the use of compounds of Formula (2), where R¹ is CH₃—, R² is H—, and R³ is H— or CH₃—. Also included is the use of compounds of Formula (2), where R¹ is CH₃—, R² is H—, and R³ is Br—. Also included is the use of compounds of Formula (2), where the compounds are in the form of salts with any pharmaceutically applicable acids or in a form of quaternized derivatives. In one use, the mammal is a human. In one use, any of the described compounds or compositions is used for the prevention of cataract. In one use, any of the described compounds or compositions is used for the prevention of alopecia.

Any pharmacological medication possessing the anti-aging (geroprotective) activity can be produced that contains an active ingredient and pharmaceutically suitable carrier, which has contains an active ingredient, any substance described by the Formula 1 or Formula 2 or any noted variation thereof.

Use of the compounds can be, but is not limited to, use for the prophylactics of untimely ageing, that can be described by giving to a patient of a pharmacological medication, which contains an effective amount of a substance described by either Formula 1 or by Formula 2 in a dose 0.1 to 10 mg/kg of the body weight, at least once a day during the period of time, which is required to reach a therapeutic effect.

### BRIEF DESCRIPTION OF THE DRAWINGS

FIG. 1 illustrates an age-dependent decline in the number of survived animals (C57/B female mice) that were receiving Dimebon in the daily dose of 3 mg/kg (experimental animals) and that were receiving pure water (control animals). The x-axis represents age in months. The y-axis represents the amount of survived animals. Diamonds represent control animals. Squares represent experimental animals.

FIG. 2 provides the body weight changes of animals (C57/B female mice) that were receiving Dimebon in the daily dose of 3 mg/kg (experimental animals) and that were receiving pure water (control animals). The x-axis represents age in months. The y-axis represents average weight in grams. Diamonds represent control animals. Squares represent experimental animals.

FIG. 3 provides data on the vision disturbances (development of the age-related cataract) in animals (C57/B female mice) that were receiving Dimebon in the daily dose of 3 mg/kg (experimental animals) and that were receiving pure water (control animals). The x-axis represents age in months. The y-axis represents the percentage of mice developing age-related cataract. Diamonds represent control animals. Squares represent experimental animals.

FIG. 4 compares the disturbances in skin-hair integument in animals (C57/B female mice) that were receiving Dimebon in the daily dose of 3 mg/kg (experimental animals) and that were receiving pure water (control animals). The x-axis represents age in months. The y-axis represents the percentage of mice developing alopecia. Diamonds represent control animals. Squares represent experimental animals.

FIGS. 5A and 5B are pictures illustrating the appearance of alopecia in mice in the control group (6A) and mice in the group that received Dimebon in a daily dose of 3 mg/kg daily in 18 months after the beginning of the experiment.

### DETAILED DESCRIPTION OF THE INVENTION

Compounds for use in any of the methods, kits, medicaments and the like described herein are hydrogenated pyrido (4,3-b) indoles or pharmaceutically acceptable salt thereof. A hydrogenated pyrido (4,3-b) indole can be a tetrahydro pyrido (4,3-b) indole. The hydrogenated pyrido (4,3-b) indole can also be a hexahydro pyrido (4,3-b) indole. The hydrogenated pyrido (4,3-b) indole compounds can be substituted with 1 to 3 substituents, although unsubstituted hydrogenated pyrido (4,3-b) indole compounds or hydrogenated pyrido (4,3-b) indole compounds with more than 3 substituents are also contemplated. Suitable substituents include but are not limited to alkyl, lower alkyl, aralkyl, heteroaralkyl, substituted heteroaralkyl, and halo.

Particular hydrogenated pyrido-([4,3-b]) indoles are exemplified by the Formula A and B:

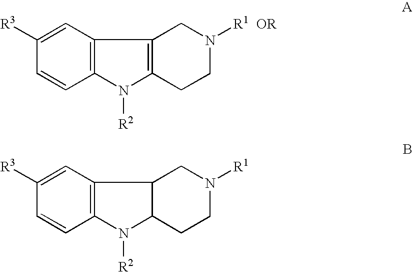

where R¹ is selected from a lower alkyl or aralkyl, R² is selected from a hydrogen, aralkyl or substituted heteroaralkyl and R³ is selected from hydrogen, lower alkyl (C1-C6alkyl) or halo.

In one variation, R1 is an alkyl group. In one variation, R1 is an aralkyl group. In one variation, R1 is an alkyl group or an aralkyl group.

In one variation, R1 is a C1-C15 alkyl. In one variation, R1 is a C10-C15 alkyl. In one variation, R¹ is a C₁-C₁₀alkyl. In one variation, R¹ is a C₁-C₈ alkyl. In one variation, R¹ is a C₁-C alkyl. In one variation, R¹ is a C₁-C₄ alkyl. In one variation, R¹ is a C₁-C₃ alkyl. In one variation, R¹ is a C₂-C₁₅ alkyl. In one variation, R¹ is a C₂-C₁₀ alkyl. In one variation, R¹ is a C₂-C₅alkyl. In one variation, R¹ is C₆-C₁₅alkyl. In one variation, R¹ is an alkyl group having more than 15 carbon atoms. In one variation, R¹ is methyl. In one variation, R¹ is ethyl. In one variation, R¹ is selected from methyl or ethyl. In one variation, R¹ is selected from methyl and an aralkyl group such as PhCH₂—. In one variation, R¹ is selected from ethyl or an aralkyl group such as PhCH₂—. In one variation, R¹ is a straight chain alkyl group of any alkyl size indicated for R¹ alkyl groups (e.g., a straight chain C₁-C₁₅ alkyl such as n-nonyl and the like). In one variation, R¹ is a branched alkyl group of any alkyl size indicated above (e.g., a branched chain C₁-C₆ alkyl such as t-butyl).

In one variation, R¹ is an aralkyl group. In one variation, R¹ is an aralkyl group where any one of the alkyl or lower alkyl substitutes listed in the immediately preceding paragraph are further substituted with an aryl group (e.g., Ar-C₁-C₆Alkyl or Ar-C₁-C₃Alkyl, Ar-C₁-C₁₅alkyl). In one variation, R¹ is an aralkyl groups where any one of the alkyl or lower alkyl substitutes listed in the immediately preceding paragraph are substituted with an aromatic carbocyclic group of from 5 to 15 carbon atoms having a single ring (e.g., phenyl) or multiple condensed rings (e.g., napthyl) which condensed rings may or may not be aromatic. In one variation, R¹ is an aralkyl group where any one of the alkyl or lower alkyl substitutes listed in the immediately preceding paragraph are further substituted with a phenyl group (e.g., Ph-C₁-C₆Alkyl or Ph-C₁-C₃Alkyl, Ph-C₁-C₁₅alkyl). In one variation, R¹ is PhCH₂—.

In one variation, R¹ is selected from an alkyl or aralkyl group, wherein the alkyl group or alkyl portion of the aralkyl moiety is a C₁-C₈ alkyl. In one variation, R¹ is selected from an alkyl or aralkyl group, wherein the alkyl group or alkyl portion of the aralkyl moiety is a C₁-C₆ alkyl. In one variation, R¹ is selected from an alkyl or aralkyl group, wherein the alkyl group or alkyl portion of the aralkyl moiety is a C₁-C₄ alkyl. In one variation, R¹ is selected from an alkyl or aralkyl group, wherein the alkyl group or alkyl portion of the aralkyl moiety is a C₁-C₃ alkyl. In one variation, R¹ is selected from an alkyl or aralkyl group, wherein the alkyl group or alkyl portion of the aralkyl moiety is a C₁-C₂ alkyl. In one variation, R¹ is selected from an alkyl or aralkyl group, wherein the alkyl group or alkyl portion of the aralkyl moiety is a C₂-C₈ alkyl. In one variation, R¹ is selected from an alkyl or aralkyl group, wherein the alkyl group or alkyl portion of the aralkyl moiety is a C₄-C₈ alkyl. In one variation, R¹ is selected from an alkyl or aralkyl group, wherein the alkyl group or alkyl portion of the aralkyl moiety is a C₆-C₈ alkyl.

In one variation, R² is H. In one variation, R² is selected from hydrogen and an aralkyl group. In one variation, R² is an aralkyl group. In one variation, R² is a substituted heteroaralkyl group. In one variation, R² is selected from hydrogen and a substituted heteroaralkyl group. In one variation, R² is selected from hydrogen, an aralkyl group and a substituted heteroaralkyl group. In one variation, R² is selected from an aralkyl group and a substituted heteroaralkyl group.

In one variation, R2 is an aralkyl group where R2 can be any one of the aralkyl groups noted for R1 above, the same as if each and every aralkyl variation listed for R1 is separately and individually listed for R2.

In one variation, R2 is a substituted heteroaralkyl group wherein an alkyl or lower alkyl group is substituted with a heteroaryl group substituted with 1 to 3 lower alkyl (C₁-C₆) substituents. In one variation, R² is a substituted heteroaralkyl group wherein an alkyl or lower alkyl group is substituted with a heteroaryl group substituted with 1 to 3 lower alkyl (C₁-C₃) substituents. In one variation, R² is a substituted heteroaralkyl group wherein an alkyl or lower alkyl group is substituted with a heteroaryl group substituted with 1 to 3 methyl groups. In one variation, R² is a substituted heteroaralkyl group wherein an alkyl or lower alkyl group is substituted with a heteroaryl group substituted with 1 lower alkyl (C₁-C₆) substituents. In one variation, R² is a substituted heteroaralkyl group wherein an alkyl or lower alkyl group is substituted with a heteroaryl group substituted with 1 lower alkyl (C₁-C₃) substituent. In one variation, R² is a substituted heteroaralkyl group wherein an alkyl or lower alkyl group is substituted with a heteroaryl group substituted with 1 to 2 methyl groups. In one variation, R² is a substituted heteroaralkyl group wherein an alkyl or lower alkyl group is substituted with a heteroaryl group substituted with 1 methyl group. R² can be any one of the substituted heteroaralkyl groups listed, where the alkyl or lower alkyl group that is substituted with a heteroaryl group is a C₁-C₁₅alkyl or a C₁-C₁₀alkyl or a C₁-C₈ alkyl or a C₁-C₁₆alkyl or a C₁-C₄alkyl or a C₂-C₄ alkyl or a C₄-C₁₀alkyl or a C₂-C₃alkyl.

In other variations, R² is any one of the substituted heteroaralkyl groups in the immediately preceding paragraph where the heteroaralkyl moiety comprises from 2 to 10 ring carbon atoms and 1 to 4 ring heteroatoms selected from oxygen, nitrogen and sulfur. In other variations, R² is any one of the substituted heteroaralkyl groups in the immediately preceding paragraph where the heteroaralkyl moiety comprises from 2 to 6 ring carbon atoms and 1 to 4 ring heteroatoms selected from oxygen, nitrogen and sulfur. In other variations, R² is any one of the substituted heteroaralkyl groups in the immediately preceding paragraph where the heteroaralkyl moiety comprises from 4 to 8 ring carbon atoms and 1 to 4 ring heteroatoms selected from oxygen, nitrogen and sulfur. In other variations, R² is any one of the substituted heteroaralkyl groups in the immediately preceding paragraph where the heteroaralkyl moiety comprises pyridyl (Py).

In one variation, R2 is 6-CH3-3-Py-(CH2)2—.

In one variation, R³ is hydrogen. In other variations, R³ is any one of the alkyl groups noted for R¹ above, the same as if each and every alkyl variation listed for R¹ is separately and individually listed for R². In another variation, R³ is a halo group. In one variation, R³ is selected from hydrogen and an alkyl group. In one variation, R³ is selected from hydrogen and a halo group. In one variation, R³ is selected from a hydrogen, alkyl or halo group. In one variation, R³ is selected from a halo and alkyl group.

In one variation, R3 is Br. In one variation, R3 is I. In one variation, R3 is F. In one variation, R3 is Cl.

In a particular variation, the hydrogenated pyrido (4,3-b) indole is 2,8-dimethyl-5-(2-(6-methyl-3-pyridyl)ethyl)-2,3,4,5-tetrahydro-1H-pyrido[4,3-b]indole (dimebon) or any pharmaceutically acceptable salt thereof.

The hydrogenated pyrido (4,3-b) indoles can be any pharmaceutically acceptable salt thereof, which are readily known to those of skill in the art. The pharmaceutically acceptable salts include pharmaceutically acceptable acid salts. Examples of particular pharmaceutically acceptable salts include hydrochloride salts or dihydrochloride salts. In a particular variation, the hydrogenated pyrido (4,3-b) indole is a pharmaceutically acceptable salt of 2,8-dimethyl-5-(2-(6-methyl-3-pyridyl)ethyl)-2,3,4,5-tetrahydro-1H-pyrido[4,3-b]indole, such as 2,8-dimethyl-5-(2-(6-methyl-3-pyridyl)ethyl)-2,3,4,5-tetrahydro-1H-pyrido[4,3-b]indole dihydrochloride.

Particular hydrogenated pyrido-([4,3-b]) indoles can also be described by the Formula (1) or by the Formula (2) as medications for the prophylactics of premature ageing, geroprotectors for mammals:

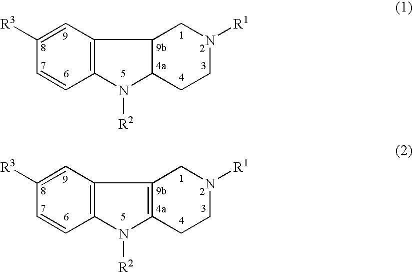

For compounds of a general Formula (1),
R¹ represents CH₃, CH₃CH₂, or PhCH₂;
R² is H, PhCH₂, or 6CH₃-3-Py-(CH₂)₂—;
R³ is H, CH₃, or Br
   in any combination of the above substituents. All possible combinations of the substituents of Formula (1) and (2) are contemplated as specific and individual compounds the same as if each single and individual compound were listed by chemical name. Also contemplated are the compounds of formula (1) or (2), with any deletion of one or more possible moieties from the substituent groups listed above: e.g., where R¹ represents CH₃; R² is H, PhCH₂, or 6CH₃-3-Py-(CH₂)₂—; and R³ is H, CH₃, or Br, or where R¹ represents CH₃; R² is 6CH₃-3-Py-(CH₂)₂—; and R³ represents H, CH₃, or Br.

The above compounds may be in a form of salt with pharmaceutically acceptable acids and in a form of quaternized derivatives.

One of the compounds, which may be used as a geroprotector, may be a compound described by a general Formula (1), where R¹ is CH₃, R² is H, and R³ is CH3.

This compound may be in a form of (±) cis-isomer.

For the compounds of a general Formula (2),
R¹ is represented by CH₃, CH₃CH₂, or PhCH₂;
R² is H, PhCH₂, or 6CH₃-3-Py-(CH₂)₂—;
R³ is H, CH₃, or Br;

The above compounds may be in a form of salt with pharmaceutically acceptable acids and in a form of quaternized derivatives.

One of compounds, which may be used as a geroprotector, may be a compound described by a general Formula (2), where R¹ is CH₃CH₂ or PhCH₂, R² is H, and R³ is H;
Or a compound, where R¹ is CH₃, R² is PhCH₂, R³ is CH;
Or a compound, where R¹ is CH₃, R² is 6-CH₃-3-Py-(CH₂)₂, and R³ is CH₃;
Or a compound, where R¹ is CH₃, R² is H, R³ is H or CH₃;
Or a compound, where R¹ is CH₃, R is H, R³ is Br.

Any of the above compounds may be used as a geroprotector in humans, in particular, for the prevention of cataract, as well as, also, in particular, for the prevention of alopecia. Other uses are described herein.

Compounds described by a general Formula (1) are known compounds, which are widely used in pharmacological practice. Broad studies were conducted with a number of known compounds, which are derivatives of tetrahydro- and hexahydro-1H-pyrido[4,3-b] indole, and which express a broad spectrum of biological activity. The following types of activity were found in a series of 2,3,4,5-tetrahydro-1H-pyrido [4,3-b] indoles: antihistamine activity (OS-DE N 1813229 from Dec. 6, 1968, N 1952800 from Oct. 20, 1969); central anti-depressant activity; anti-inflammatory activity (U.S. Pat. No. 3,718,657 from Dec. 13, 1970); neuroleptic activity (Herbert, C. A., Plattner, S. S., Welch, W. N., Mol. Pharm., 1980, v. 17, N. 1, p. 38-42) and other types of activity. Derivatives of 2,3,4,4a,5,9b-hexahydro-1H-pyrido[4,3-b]indole express psychotropic activity {Welch, W. H., Herbert, C. A., Weissman, A., Koe, K. B., J. Med. Chem., 1986, vol. 29, N 10, p. 2093-2099), anti-aggressive activity, antiarrhythmic activity, and other types of activity.

The following therapeutic drugs, which are derivatives of tetrahydro- and hexahydro-1H-pyrido[4,3-b]indole, are manufactured: “diazoline” (mebhydroline), dimebon, “dorastine”, “carbidine” (“dicarbine”), “stobadine”, “gevotroline”. Diazoline (2-methyl-5-benzyl-2,3,4,5-tetrahydro-1H-pyrido[4,3-b]indole dihydrochloride) (Klyuev, M. A. Therapeutic drugs, which are approved in the medicinal practice in the USSR.—Moscow, Medicina, 1991, p. 512) and dimebon (2,8-dimethyl-5-(2-(6-methylpyridyl-3)ethyl)-2,3,4,5-tetrahydro-1H-pyrido [4,3-b]indole dihydrochloride) (Mashkovsky, M. D. Medicinal drugs. Part 1, 12^{th} edition, Moscow, Medicine, 1993, p. 383), and its close analogue dorastine (2-methyl-8-chloro-5-(2-(6-methyl-3-pyridyl)ethyl)-2,3,4,5-tetrahydro-1H-pyrido[4,3-b]indole dihydrochloride) (USAN and USP dictionary of drug names (United States Adopted Names 1961-1988, current US Pharmacopoeia and National Formulae for Drugs, and other nonproprietary drug names) 1989, 26^{th} edition, p. 196) are known as antihistamine medications. Carbidine (dicarbine) (cis(±)2,8-dimethyl-2,3,4,4a,5,9b-hexahydro-1H-pyrido[4,3-b]indole dihydrochloride) is a Russian neuroleptic drug, which also has an antidepressive effect (Yakhontov, L. N., Glushkov, R. G. Synthetic therapeutic drugs. A. G. Natradze, editor, Moscow Medicina, 1983, p. 234-237). Stobadine, a (−) isomer of carbidine, is known as an anti-arrhythmic medication (Kitlova, M., Gibela, P., Drimal, J. Bratisi. Lek. Listy, 1985, vol. 84, N 5, p. 542-546). Gevotroline (8-fluoro-2-(3-3-pyridyl)propyl-2,3,4,5-tetrahydro-1H-pyrido[4,3-b]indole dihydrochloride) is an anti-psychotic and an anxyolytic medication (Abou-Gharbi, M., Patel, U. R., Webb, M. B., Moyer, J. A., Ardnee, T. H., J. Med. Chem., 1987, v.30, p. 1818-1823). Regarding dimebon and other compounds, see also Galenki-Iaroshevskii, P. A., Melkumove, E. R.; Bartahevich, V. V., Uvarov, A. V., Turovaia, A,; Khankoeva, A. T., Galygo, D. S. (1996) Biull Eksp Biol Med. (Russ), 122(12):642-644; and Shevtova, E., Kireeva, E., Lermontova, N., Bachurin, S, Abstract, 2^{nd} Colloquium on Mitochondria and Myopathies in Halle/Saale, Mar. 31-Apr. 2, 2000, “Beta-Amyloid Peptide (25-35) as the Trigger of Mitochondrial Permeability Transition”.

More recently, it was found that derivatives of hydrogenated pyrido[4,3-b]indoles described by the Formula (1) and Formula (2), e.g., dimebon, also possess properties of antagonists of NMDA receptors, that makes these compounds useful for the therapy of neurodegenerative diseases, and especially, Alzheimer's disease (patent Russian Federation No. 2106864 C1 6 A 61 K 31/475, Mar. 20, 1998, bull. 8; U.S. Pat. No. 6,187,785).

All compounds mentioned above are known from literature and include the following specific compounds:
1. cis(±) 2,8-dimethyl-2,3,4,4a,5,9b-hexahydro-1H-pyrido[4,3-b]indole and its dihydrochloride;
2. 2-ethyl-2,3,4,5-tetrahydro-1H-pyrido[4,3-b]indole;
3. 2-benzyl-2,3,4,5-tetrahydro-1H-pyrido[4,3-b]indole;
4. 2,8-dimethyl-5-benzyl-2,3,4,5-tetrahydro-1H-pyrido[4,3-b]indole and its dihydrochloride;
5. 2-methyl-5-(2-methyl-3-pyridyl)ethyl-2,3,4,5-tetrahydro-1H-pyrido[4,3-b]indole and its sesquisulfate;
6. 2,8-dimethyl-5-(2-(6-methyl-3-pyridyl)ethyl)-2,3,4,5-tetrahydro-1H-pyrido[4,3-b]indole and its dihydrochloride (dimebon);
7. 2-methyl-2,3,4,5-tetrahydro-1H-pyrido[4,3-b]indole;
8. 2,8-dimethyl-2,3,4,5-tetrahydro-1H-pyrido[4,3-b]indole and its methyl iodide;
9. 2-methyl-8-bromo-2,3,4,5-tetrahydro-1H-pyrido[4,3-b]indole and its hydrochloride.

Synthesis and studies on neuroleptic properties for the compound 1 are reported, for instance, in the following publication: Yakhontov, L. N., Glushkov, R. G., Synthetic therapeutic drugs. A. G. Natradze, the editor, Moscow Medicina, 1983, p. 234-237. Synthesis of compounds 2, 8, and 9, and data on their properties as serotonin antagonists are reported in, for instance, in C. J. Cattanach, A. Cohen & B. H. Brown in J. Chem. Soc. (Ser. C) 1968, p. 1235-1243. Synthesis of the compound 3 is reported, for instance, in the article N. P.Buu-Hoi, O. Roussel, P. Jacquignon, J. Chem. Soc., 1964, N 2, p. 708-711. N. F. Kucherova and N. K. Kochetkov (General chemistry (russ.), 1956, v. 26, p. 3149-3154) describe the synthesis of the compound 4. Synthesis of compounds 5 and 6 is described in the article by A. N. Kost, M. A. Yurovskaya, T. V. Mel'nikova, in Chemistry of heterocyclic compounds, 1973, N 2, p. 207-212. The synthesis of the compound 7 is described by U. Horlein in Chem. Ber., 1954, Bd. 87, hft 4, 463-p. 472. M. Yurovskaya and I. L. Rodionov in Chemistry of heterocyclic compounds (1981, N 8, p. 1072-1078) describe the synthesis of methyl iodide of the compound 8.

Unexpectedly, as discussed in Example 1, it was found that compounds described by the Formula (1) and Formula (2) have an anti-calcium activity, in particular it was found that these compounds are capable of inhibiting the entry of calcium ions into the nerve cells, which was induced by the activation of glutamate receptors. On the other hand, it is known that hyperaccumulation of calcium ions in cells induces a cascade of degenerative processes, which accompany the process of ageing and a development of corresponding degenerative diseases. A disturbance in calcium homeostasis is the basis for a so-called calcium theory of ageing and dementia (Calcium Hypothesis of Ageing and Dementia. Ann. N.Y. Acad. Sci., 1994, v. 747).

As described in the Examples, it was also discovered that a compound, dimebon, slowed aging, prolonged life, reduced occurrence of hair loss, and reduced cataracts in aging mice. Thus, because of the new unexpected properties which do not follow from the structure of compounds described by Formula (1) and Formula (2) as well as other compounds described herein, such as compounds described by Formula (A) and (B), these compounds may be used as geroprotectors.

According to this invention, a pharmacological tool, which has a geroprotective activity, and which contains an active ingredient and a pharmaceutically acceptable carrier, has an effective amount of hydrogenated pyrido[4,3-b]indole described by the Formula (1) or by Formula (2) as an active ingredient. In other embodiments is a pharmacological tool, which has a geroprotective activity, and which contains an active ingredient and a pharmaceutically acceptable carrier, has an effective amount of hydrogenated pyrido[4,3-b]indole described by the Formula (A) or by Formula (B) as an active ingredient.

The definition “geroprotective activity”, which is used in the present application, means a biological activity that slows down ageing and/or prolongs life and/or increases or improves -the quality of life via a decrease in the amount and/or the level of intensity of pathologies or conditions that are not life-threatening but are associated with the aging process and which are typical for elderly people. Pathologies or conditions that are not life-threatening but are associated with the aging process include such pathologies or conditions as loss of sight (cataract), deterioration of the dermatohairy integument (alopecia), and an age-associated decrease in weight due to the death of muscular and/or fatty cells.

The definition “pharmacological tool” means the use of any therapeutic form that contains a compound described by the Formula (1) or by Formula (2), which may be useful for the prophylactic or for the therapeutic application in medicine as a tool with a geroprotective activity for the prophylactics of ageing. In order to make a pharmacological tool, one or several compounds described by a Formula (1) or by Formula (2) as an active ingredient is mixed with a pharmacologically acceptable carrier, which is known in medicine, according to the acceptable pharmaceutical method. Depending on the therapeutic form of the medication, the carrier may be in various forms. This disclosure also provides additional pharmacological tools related to compound of Formula (1) or (2), for example, pharmacological tools related to compounds of Formula (A) or (B).

The definition “effective amount”, which is used in the present application means the use of such amount of a compound described by the Formula (1) or by Formula (2) or any compound described herein, such as any compound described by the Formula (A) or (B), which in combination with its parameters of efficacy and toxicity, as well as based on the knowledge of the practicing specialist should be effective in a given therapeutic form. As is understood in the art, an effective amount may be in one or more doses.

The present invention provides a variety of methods, such as those described in the “Brief Summary of the Invention” and elsewhere in this disclosure. The methods of the invention employ the compounds described herein. For example, in one embodiment, the present invention provides a method of prolonging the lifespan of an individual comprising administering to an individual an effective amount of a hydrogenated pyrido (4,3-b) indole or pharmaceutically acceptable salt thereof. The present invention also provides a method of prolonging the lifespan of cells in an individual comprising administering to an individual an effective amount of a hydrogenated pyrido (4,3-b) indole or pharmaceutically acceptable salt thereof. The pharmacological tool (or compounds) described herein can also be used to slow aging in an individual. For example, the pharmacological tools (or compounds) can be used for delaying the onset and/or slowing the progression of an aging-associated manifestation and/or pathology or condition, including, but not limited to, any one or more of: disturbance in skin-hair integument (such as baldness or alopecia), visual disturbance (such as development of cataracts), and weight loss (including weight loss due to the death of muscular and fatty cells). The compounds can also be used to improve the quality of life of an individual, such as an individual developing these age-associated manifestations and/or pathologies. The compounds can also be used to decrease the risk of developing an age-related condition, such as a non life-threatening age-related pathology or condition.

For use herein, unless clearly indicated otherwise, use of the terms “a”, “an” and the like refers to one or more.

Mammals (interchangeably referred to as “individuals” herein) include, but are not limited to, human, bovine, primate, equine, canine, feline, porcine, and ovine animals. Thus, the invention finds use, for example, in the veterinary context, such as for use in agriculture and domestic pets. The individual may manifest one or more signs of aging, such as hair loss (including baldness), wrinkles, grey hair, and weight loss (including weight loss due to the death of muscular and/or fatty cells). In some embodiments, the individual is a mammal for which slowing aging is desired. In some embodiments, the individual is a human who desires to slow aging. In some embodiments, the individual is an individual in need of any of the methods described herein. The individual may be a human who is at least 35 years old and/or less than about 60 years old. In some embodiments, the compound is administered to a human who is at least about 40, about 45, about 50, about 55, about 60, about 65, about 70, about 75, about 80, about 85 years old. In some embodiments, the compound is administered to a human who is between about 40 and about 100 years old, in another embodiment, the human is from about 40 and about 80 years old. In another embodiment, the human is from about 40 to about 60 years old. In another embodiment, the human is between about 60 to 100 years old. In another embodiment, the human is between about 60 to 80 years old. In another embodiment, the human is between about 50 and 70 years old. In another embodiment, the human is between about 70 to 90 years old. The individual may be a human who has not been diagnosed with neurological diseases (such as Alzheimer's disease) or a cognition impairment associated with aging. The individual may be a human who does not display a symptom of cognitive impairment. In some embodiments, the individual is in need of any one or more of the methods for ameliorating one or more manifestations of aging (also referred to as aging-associated manifestations) described herein. In some embodiments, the individual is other than a human.

According to the present invention, methods of the present invention (such as a method slowing aging may comprise the administration to an individual (such as a human patient) of the pharmacological tool that contains the effective amount of hydrogenated pyrido[4,3-b]indoles described by the Formula (1) or by Formula (2) or any other hydrogenated pyrido[4,3-b]indoles described herein, such as those described in Formula (A) and (B), in dose of between 0.1 and 10 mg/kg of the body weight, at least once a day and during the period of time, which is required to achieve the therapeutic effect. In other variations, the daily dose (or other dosage frequency) of a hydrogenated pyrido[4,3-b]indole as described herein is between about 0.1 and about 8 mg/kg; or between about 0.1 to about 6 mg/kg; or between about 0.1 and about 4 mg/kg; or between about 0.1 and about 2 mg/kg; or between about 0.1 and about 1 mg/kg; or between about 0.5 and about 10 mg/kg; or between about 1 and about 10 mg/kg; or between about 2 and about 10 mg/kg; or between about 4 to about 10 mg/kg; or between about 6 to about 10 mg/kg; or between about 8 to about 10 mg/kg; or between about 0.1 and about 5 mg/kg; or between about 0.1 and about 4 mg/kg; or between about 0.5 and about 5 mg/kg; or between about 1 and about 5 mg/kg; or between about 1 and about 4 mg/kg; or between about 2 and about 4 mg/kg; or between about 1 and about 3 mg/kg; or between about 1.5 and about 3 mg/kg; or between about 2 and about 3 mg/kg; or between about 0.01 and about 10 mg/kg; or between about 0.01 and 4 mg/kg; or between about 0.01 mg/kg and 2 mg/kg; or between about 0.05 and 10 mg/kg; or between about 0.05 and 8 mg/kg; or between about 0.05 and 4 mg/kg; or between about 0.05 and 4 mg/kg; or between about 0.05 and about 3 mg/kg; or between about 10 kg to about 50 kg; or between about 10 to about 100 mg/kg or between about 10 to about 250 mg/kg; or between about 50 to about 100 mg/kg or between about 50 and 200 mg/kg; or between about 100 and about 200 mg/kg or between about 200 and about 500 mg/kg; or a dosage over about 100 mg/kg; or a dosage over about 500 mg/kg. In some embodiments, a daily dosage of Dimebon is administered. The daily dosage for Dimebon can be a 10 mg/kg dosage.

The compound may be administered for a sustained period, such as at least about one month, at least about 2 months, at least about 3 months, at least about 6 months, or at least about 12 months or longer.

Other dosing schedules may also be followed. For example, the frequency of the administration may vary. The dosing frequency can be a once weekly dosing. The dosing frequency can be a once daily dosing. The dosing frequency can be more than once weekly dosing. The dosing frequency can be more than once daily dosing, such as any one of 2, 3, 4, 5, or more than 5 daily doses. The dosing frequency can be 3 times a day. The dosing frequency can be three times a week dosing. The dosing frequency can be a four times a week dosing. The dosing frequency can be a two times a week dosing. The dosing frequency can be more than once weekly dosing but less than daily dosing. The dosing frequency can be a once monthly dosing. The dosing frequency can be a twice weekly dosing. The dosing frequency can be more than once monthly dosing but less than one weekly dosing. The dosing frequency can intermittent (e.g., one daily dosing for 7 days followed by no doses for 7 days, repeated for any 14 day time period, such as 2 months, 4 months, 6 months or more). The dosing frequency can be continuous (e.g., one weekly dosing for continuous weeks). Any of the dosing frequencies can be used with any dosage amount, for example, any of the of dosing frequencies can employ a 10 mg/kg dosage amount. Any of the dosing frequencies can employ any of the compounds described herein together with any of the dosages described herein, for example, the dosing frequency can be a three times daily 10 mg/kg dose of dimebon.

Compounds described by Formula (1) or by Formula (2) or compounds described by Formula (A) or (B) may be administered to mammals in a form of generally accepted oral compositions, such as tablets, coated tablets, gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical preparations may contain preservatives, solubilizers, stabilizers, re-wetting agents, emulgators, sweeteners, dyes, adjusters, salts for the adjustment of osmotic pressure, buffers, coating agents or antioxidants. Preparations may also contain other substances, which have valuable therapeutic properties. Therapeutic forms may be represented by a usual standard dose and may be prepared by a known pharmaceutical method. Suitable formulations can be found, e.g., in Remington 's Pharmaceutical Sciences, Mace Publishing Company, Philadelphia, Pa., 20^{th} ed. (2000), which is incorporated herein by reference. Any of the compounds described herein can be formulated in a tablet in any dosage form described, for example, dimebon or a pharmaceutically acceptable salt thereof can be formulated as a 10 mg tablet. Any of the compounds described herein can be formulated in any dosage as a sustained release formulation. The invention also provides for a sustained release devise, for example a transdermal patch or an implantable devise comprising as the active ingredient any one of the compounds described herein in any total amount such that the individual receives an effective amount of compound during the sustained release period. The technical result, that may be achieved after the application of the present invention, may be a slowing of aging, and/or a significant prolongation of life, and/or an improvement of the quality of life via a decrease in the amount and/or the level of intensity of pathologies or conditions that are not life-threatening but are associated with the aging process, such as loss of sight (cataract), deterioration of the dermatohairy integument (alopecia), an age-associated decrease in weight due to the death of muscular and/or fatty cells.

The invention further provides kits for carrying out the methods of the invention, which comprises one or more compounds described herein. The kits may employ any of the compounds disclosed herein. In one variation, the kit employs dimebon or a pharmaceutically acceptable salt thereof, such as the dihydrochloride salt. The kits may be used for any one or more of the uses described herein, and, accordingly, may contain instructions for any one or more of the following uses: slowing aging (such as delaying the onset or slowing the progression of an age-associated or age-related manifestation and/or pathology or condition), prolonging lifespan of an individual, prolonging lifespan of cells in an individual, improving quality of life of an individual, and decreasing risk of developing an age-related condition, such as a non-life-threatening age-related pathology or condition.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention also provides compositions (including pharmaceutical compositions) as described herein for the use of any of slowing aging, prolonging lifespan, and other methods described herein.

A possibility of realization of the present invention with the achievement of the claimed goal and the achievement of the technical result is supported by not restricted by the following data.

### EXAMPLES

### Example 1

### Determination of Calcium Blocking Properties of Hydrogenated pyrido (4,3-b) indoles of Formula (1) and Formula (2)

Evaluation of the calcium-blocking properties of the compounds was conducted with P2-fraction of synaptosomes, which were isolated from the brain of newborn (8-11 days) rats according to the protocol described in [Bachurin et al. Neuroprotective and cognition enhancing properties of MK-801 flexible analogs. Structure-activity relationships.//Ann. N.Y. Acad. Sci., 2001, v. 939, pp. 219-235]. In this test, the ability of the compounds to inhibit a specific uptake of calcium ions via ion channels associated with glutamate receptors was determined.

Synaptosomes were placed into the incubation buffer A (132 mM NaCl, 5 mM KCl, 5 mM HEPES) and were kept at 0° C. during the entire experiment. Aliquots of synaptosomes (50 μl) were placed in the media A, containing investigated compounds and a preparation of the radiolabeled calcium, ⁴⁵Ca. The calcium uptake was stimulated by the introduction into the media of the 20 μl of the 10 mM solution of glutamate. After a 5 min incubation at 30° C. the reaction was interrupted by a filtration through GF/B filters, which were then triple washed with the cold buffer B (145 mM KCl, 10 mM tris, 5 mM trilon B). Then, filters were subjects for the detection of the radiolabeled calcium. The measurement was conducted using a scintillation counter ‘SL-4000 Intertechnic”. The initial screening was conducted with a concentration of each compound of 5 μM. A specific calcium uptake was calculated using the following equation

K(43/21)=[(Ca4−Ca3)/(Ca2−Ca1)]*100%

where:
Ca1 is a calcium uptake in a control experiment (no glutamate or test compound added);
Ca2 is a calcium uptake in the presence of glutamate only (Glutamate Induced Calcium Uptake—GICU);
Ca3 is a calcium uptake in the presence of a test compound only (no glutamate added); Ca4 is a calcium uptake in the presence of both glutamate and a test compound.

Results of these experiments are presented in Table 1.

TABLE 1
Effect of investigated compounds on the glutamate-induced
uptake of ⁴⁵Ca into the rat brain synaptosomes.

| | | % Ca to |
|---|---|---|
| # | Compound | control |
| 1 | cis-(±)-2,8-dimethyl-2,3,4,4a,5,9b-hexahydro- | 40.5 ± 1.0 |
| | 1H-pyrido(4,3-b)indol dihydrochloride | |
| 2 | 2-ethyl-2,3,4,5-tetrahydro-1H-pyrido(4,3-b)indol | 36.5 ± 0.5 |
| 3 | 2-benzyl-2,3,4,5-tetrahydro-1H-pyrido(4,3-b)indol | 28.5 ± 2.5 |
| 4 | 2,8-dimethyl-5-benzyl-2,3,4,5-tetrahydro-1H- | 22.5 ± 3.5 |
| | pyrido(4,3-b)indol dihydrochloride | |
| 5 | 2-methyl-5-(2-(6-methyl-3-pyridyl)-ethyl)- | 33.5 ± 1.5 |
| | 2,3,4,5-tetrahydro-1H-pyrido(4,3-b)indol | |
| | sesquisulfate monohydrate | |
| 6 | 2,8-dimethyl-5-(2-(6-methyl-3-pyridyl)-ethyl)- | 48.5 ± 2.5 |
| | 2,3,4,5-tetrahydro-1H-pyrido(4,3-b)indol | |
| | dihydrochloride (Dimebon) | |
| 7 | 2-methyl-2,3,4,5-tetrahydro-1H-pyrido(4,3- | 35.5 ± 2.5 |
| | b)indol | |
| 8 | 2-dimethyl-2,3,4,5-tetrahydro-1H-pyrido(4,3- | 18.0 ± 2.5 |
| | b)indol methyl iodide | |
| 9 | 2-methyl-8-bromo-2,3,4,5-tetrahydro-1H-pyrido(4,3- | 30.0 ± 3.5 |
| | b)indol hydrochloride | |

Table 1 illustrates that all investigated compounds possess pronounced calcium-blocking properties. This suggests that according to the described above ageing and dementia hypothesis, Calcium hypothesis of Ageing and Dementia. Ann. N.Y. Acad. Sci., 1994, v. 747, all these compound may have a potential as geroprotectors.

### Example 2

### Data on the Activity of Dimebon as a Geroprotector

Dimebon, 2,8-dimethyl-5-(2-(6-methyl-3-pyridyl)-ethyl)-2,3,4,5-tetrahydro-1H-pyrido(4,3-b)indol dihydrochloride, was used as a representative for the compounds of a general Formula (2)

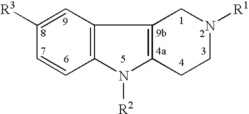

×2 HCl
where R¹ and R³ are methyls, and
R² is 2-(6-methyl-3-pyridyl)-ethyl
   Dimebon was evaluated as an agent that prolongs life and improves the quality of life (characterized by changes in the amount of pathologies that accompany ageing) in the laboratory animals.

Experiments were conducted with C57/B female mice, starting from their age of 12 months. Mice were kept on cells, 10 animals per a cell. Both control and experimental group included 50 animals in each group. Animals had a free access to food and water. The day-night cycle was 12 hours.

Prior to the experiment, daily and weekly water consumption by the animals in one cell was measured. Dimebon was added in water in such amount that each animal would consume 3 mg/kg of Dimebon per day in average. Bottles with water containing Dimebon were replaced every 7 days. Animals in the control group were receiving pure water.

Prior to the experiment, all the animals were weighed, and an average weight was determined in every group, every cell, as well as the weight of all animals in every cell. The condition of skin, hair, and eyes were also determined by visual inspection. All animals appeared healthy and did not have any visible lesions prior to the experiment. Evaluation of all these parameters was conducted on a monthly basis.

Statistics was calculated using Student T-test and ‘ Chi-squared’ criteria.

### Results

### Lifespan

Evaluation of the parameter of the length of life was conducted employing methods used in demography. This parameter was a probability of death in every age group. Results of this study are presented in Table 2.

TABLE 2
Amount of alive animals (C57/B female mice), which receive either
Dimebon in a daily dose of 3 mg/kg (experimental group) or pure
water (control group) depending on the age of the animals.

| | Age, months | | | | | | | | | | | | | |
|---|---|---|---|---|---|---|---|---|---|---|---|---|---|---|
| | 12 | 13 | 14 | 15 | 16 | 17 | 18 | 19 | 20 | 21 | 22 | 23 | 24 | 25 |
| Experimental | 50 | 50 | 49 | 48 | 47 | 47 | 42 | 39 | 35 | 32 | 28 | 25 | 18 | 11 |
| group, | | | | | | | | | | | | | | |
| amount | | | | | | | | | | | | | | |
| Control | 50 | 50 | 50 | 46 | 42 | 41 | 37 | 36 | 22 | 22 | 19 | 15 | 11 | 6 |
| group, | | | | | | | | | | | | | | |
| amount | | | | | | | | | | | | | | |

The top row is the age of animals in months. The middle and the bottom rows are numbers of alive animals.

Table 2 illustrates that death of animals started from the 14^{th} month, i.e. 2 months after the beginning of the experiment. During the entire experiment (except for the 14^{th} month) the number of animals in the experimental group was greater than in the control group. In other words, the probability of death was lower in all age groups in animals that were receiving Dimebon. In age groups of 20-23 months this difference was statistically significant (P<0.05).

### Dynamics in Weight of Animals

A decrease in the animal weight was observed during the entire experiment in the control group. This is a natural process, which is known as an age-related weight depletion. No weight depletion was observed in the group of animals that were treated with Dimebon (table 3). The depletion in weight in the experimental group was observed only in the 23 month old animals, however, even then, their weight was higher compared to the animals in the control group. It should be noted, though, that the observed variation in weight was not statistically significant (P>0.05).

TABLE 3
Dynamics in weight (g) in mice depending on age and on a Dimebon consumption.

| | Age, months | | | | | | | | | | | | | |
|---|---|---|---|---|---|---|---|---|---|---|---|---|---|---|
| | 12 | 13 | 14 | 15 | 16 | 17 | 18 | 19 | 20 | 21 | 22 | 23 | 24 | 25 |
| Exp. | 24.7 | 24.7 | 24.7 | 26 | 25.5 | 24.7 | 25.5 | 26 | 26 | 25.6 | 25.1 | 24.7 | 24 | 23.7 |
| group | | | | | | | | | | | | | | |
| Control | 25.3 | 24.7 | 24.3 | 24.6 | 24.5 | 23.5 | 24.2 | 23.5 | 23.8 | 23.2 | 23.7 | 23 | 22.8 | 22.2 |
| group | | | | | | | | | | | | | | |

The top row is the age of animals in months, The middle and the bottom rows are the average weight of animals in grams.

### Vision Disturbances

Vision disturbances, appearing as a development of cataract on one or both eyes, was observed in the control group of animals on the second month of the experiment (photo 1). The amount of animals with cataract in this group was rapidly growing every month. The amount of animals that had cataract in the group receiving Dimebon (table 4) was significantly less (P<0.05, for 13 to 20 months old mice).

TABLE 4
Vision disturbances (development of age-related cataract)
in mice depending on age and on a Dimebon consumption.

| | Age, months | | | | | | | | | |
|---|---|---|---|---|---|---|---|---|---|---|
| | 12 | 13 | 14 | 15 | 16 | 17 | 18 | 19 | 20 | 21 |
| Exp. | 0 | 0 | 0 | 4 | 6 | 6 | 10 | 13 | 17 | 25 |
| group | | | | | | | | | | |
| Control | 0 | 4 | 4 | 11 | 19 | 30 | I.D. | I.D. | I.D. | I.D. |
| group | | | | | | | | | | |

The top row is the age of animals in months. The middle and the bottom rows are the amount of animals that have cataract, in %, I.D.—insufficient data.

Starting with the month 18, the amount of animals having cataract also increased in the experimental group. The comparative analysis between the control and experimental groups between months 18 through 21 was complicated, because many of the animals that had cataract in the control group died, while animals in the experimental group were still alive.

### Skin and Hair Condition

Starting from the first month of the experiment, animals with the disturbances in their skin-hair integument, in the form of bald spots or so-called alopecia, were observed (photo). The size of bald spots in these animals was varied from 1 to 25% of the body surface. The difference in the amount of animals that had alopecia in the control and in the experiment groups was observed starting from the 13^{th} through 21^{st} month (table 5). The difference was statistically significant (P<0.05).

TABLE 5
Disturbances in skin-hair integument in mice depending
on age and on a Dimebon consumption.

| | Age, months | | | | | | | | | |
|---|---|---|---|---|---|---|---|---|---|---|
| | 12 | 13 | 14 | 15 | 16 | 17 | 18 | 19 | 20 | 21 |
| Exp. | 0 | 0 | 2 | 6 | 10 | 20 | 12 | 14 | 34 | 35 |
| group | | | | | | | | | | |
| Control | 0 | 2 | 2 | 13 | 21 | 24 | 32 | 37 | 55 | 60 |
| group | | | | | | | | | | |

The top row is the age of animals in months. The middle and the bottom rows are the % of animals that have alopecia.

By the end of the experiment, same as in case with cataract, the amount of animals that had alopecia in the control group declined because of the death of animals, while animals in the experimental group were still alive.

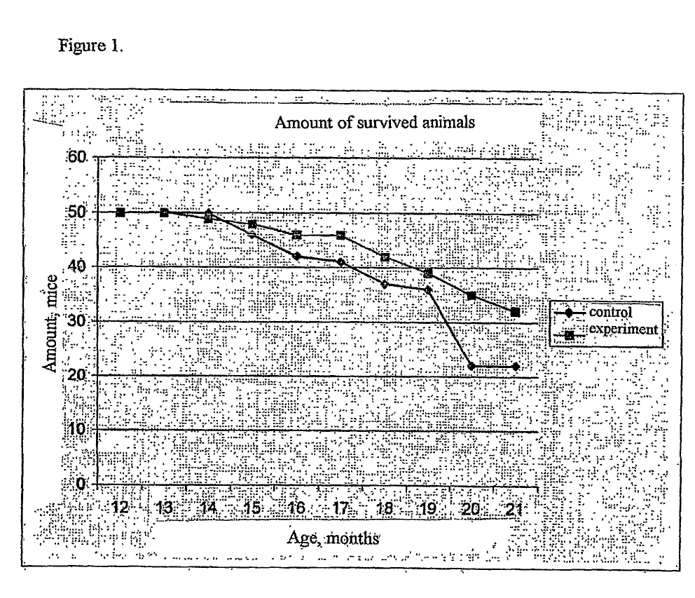

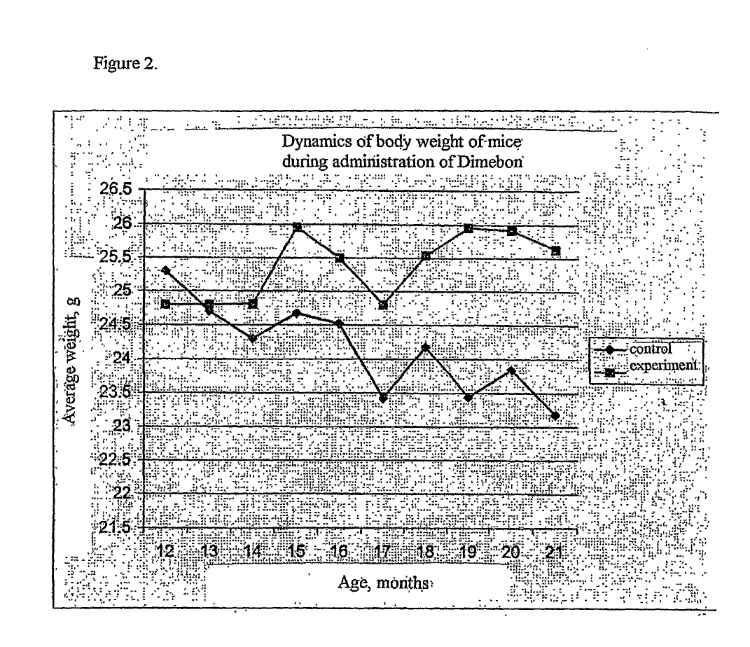

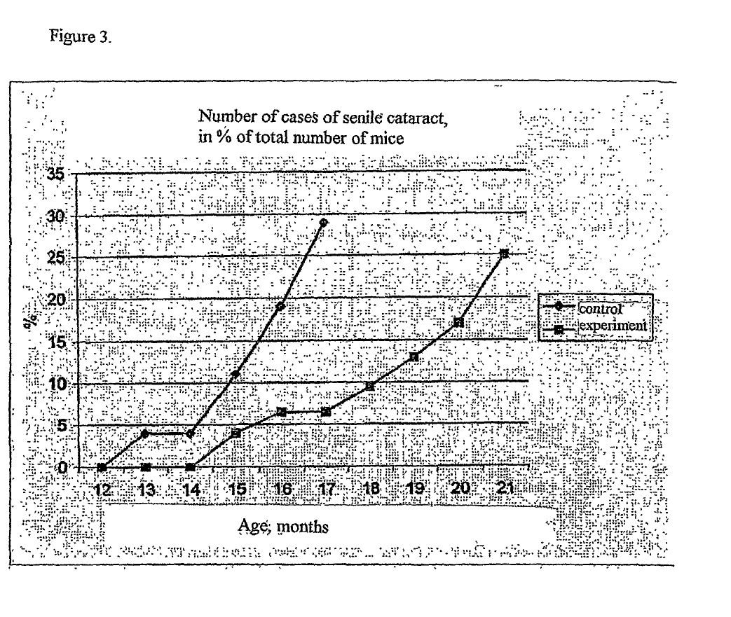

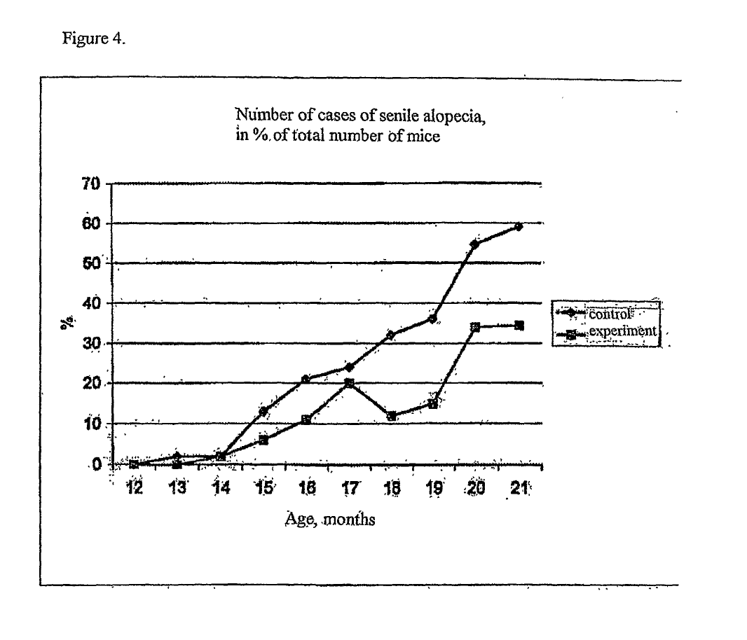

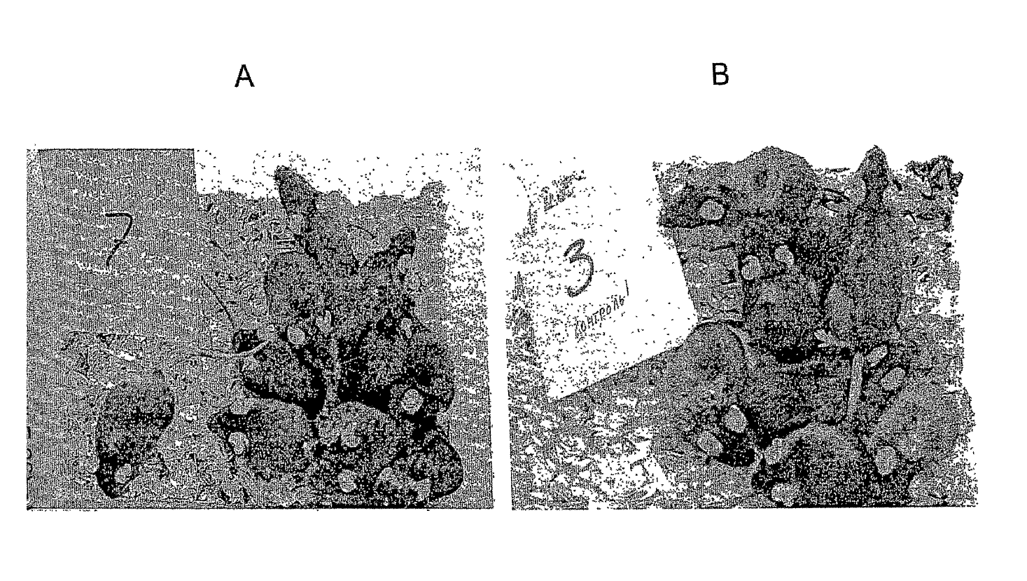

Data presented in Tables 2-5 are also presented as plots on FIGS. 1-5 for the visualization. FIG. 1 illustrates an age dependent decline in the amount of animals (C57/B female mice) that were receiving Dimebon in the daily dose of 3 mg/kg (experimental animals) and that were receiving pure water (control animals). FIG. 2 presents data on the weight dynamics in mice depending on age and a Dimebon consumption. FIG. 3 presents data on the vision disturbances (development of the age-related cataract) in mice depending on age and a Dimebon consumption. FIG. 4 illustrates the disturbances in skin-hair integument in mice depending on age and a Dimebon consumption. FIGS. 5A and 5B illustrate the appearance of alopecia of mice in the control group (A) and mice in the group that received Dimebon in a daily dose of 3 mg/kg in 18 months after the beginning of the experiment.

The presented results suggest that Dimebon statistically reliably decreases the probability of death in old animals. In addition, there is a statistically reliable evidence that there is a strong correlation between feeding old animals with Dimebon and slowing of the development of non-fatal pathologies, such as loss of vision and alopecia. A decrease in weight, which is a valid characteristic of ageing, is significantly slower in a group receiving Dimebon compared to the control group. Thus, Dimebon prolongs life and decreases the probability of non-fatal age-related pathologies. In other words, it improves the quality of life of old animals. All of the above suggests that Dimebon, in addition to its ability to have a potential in a therapy of Alzheimer's disease is an effective geroprotector.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method of slowing aging in a mammal, the method comprising administering to a mammal an amount of a hydrogenated pyrido (4,3-b) indole or pharmaceutically acceptable salt thereof effective to slow aging.

2-18. (canceled)

19. A method of slowing the progression of age associated hair loss in a mammal, the method comprising administering to a mammal an amount of a hydrogenated pyrido (4,3-b) indole or pharmaceutically acceptable salt thereof effective to slow the progression of age associated hair loss.

20. The method of claim 19, wherein the hydrogenated pyrido (4,3-b) indole is a tetrahydro pyrido (4,3-b) indole.

21. The method of claim 19, wherein the hydrogenated pyrido (4,3-b) indole is a hexahydro pyrido (4,3-b) indole.

22. The method of claim 19, wherein the hydrogenated pyrido (4,3-b) indole is of the formula: 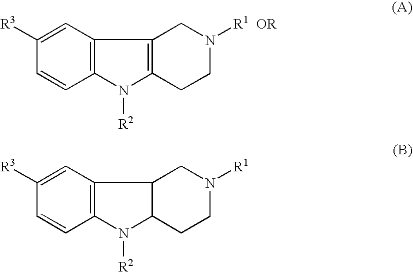
wherein:
R¹ is selected from a lower alkyl or aralkyl
R² is selected from a hydrogen, aralkyl or substituted heteroaralkyl
R³ is selected from hydrogen, lower alkyl or halo.

23. The method of claim 22, wherein aralkyl is PhCH₂— and substituted heteroaralkyl is 6-CH₃-3-Py-(CH₂)₂—.

24. The method of claim 22, wherein
R₁ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—.

25. The method of claim 19, wherein the hydrogenated pyrido (4,3-b) indole is selected from the group consisting of:
cis(±) 2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido [4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole;
2-methyl-8-bromo-2,3,4,5-tetrahydro-1H-pyrido[4,3-b]indole.

26. The method of claim 25, wherein the hydrogenated pyrido (4,3-b) indole is 2,8-dimethyl-5-(2-(6-methyl-3-pyridyl)ethyl)-2,3,4,5-tetrahydro-1H-pyrido[4,3-b]indole.

27. The method of claim 19 or 26, wherein the pharmaceutically acceptable salt is a pharmaceutically acceptable acid salt.

28. The method of claim 19, wherein the pharmaceutically acceptable salt is a hydrochloride acid salt.

29. The method of claim 19, wherein the hydrogenated pyrido (4,3-b) indole is 2,8-dimethyl-5-(2-(6-methyl-3-pyridyl)ethyl)-2,3,4,5-tetrahydro-1H-pyrido[4,3-b]indole dihydrochloride.

30. The method of claim 24, wherein R¹ is CH₃—, R² is H and R³ is CH₃—.

31. The method of claim 24 wherein R¹ CH₃CH₂— or PhCH₂—, R² is H—, and R³ is CH₃—.

32. The method of claim 24, wherein R¹ is CH₃—, R² is PhCH₂—, and R³ is CH₃—.

33. The method of claim 24, wherein R¹ is CH₃—, R² is 6-CH₃-3-Py-(CH₂)₂—, and R³ is H—.

34. The method of claim 24, where R² is 6-CH₃-3-Py-(CH₂)₂—.

35. The method of claim 24, wherein R¹ is CH₃—, R² is H—, and R³ is H— or CH₃—.

36. The method of claim 24, where R¹ is CH₃—, R² is H—, and R³ is Br—.

37. A method of slowing the progression of age associated weight loss in a mammal, the method comprising administering to a mammal an amount of a hydrogenated pyrido (4,3-b) indole or pharmaceutically acceptable salt thereof effective to slow the progression of age associated weight loss.

38. The method of claim 37, wherein the hydrogenated pyrido (4,3-b) indole is a tetrahydro pyrido (4,3-b) indole.

39. The method of claim 37, wherein the hydrogenated pyrido (4,3-b) indole is a hexahydro pyrido (4,3-b) indole.

40. The method of claim 37, wherein the hydrogenated pyrido (4,3-b) indole is of the formula: 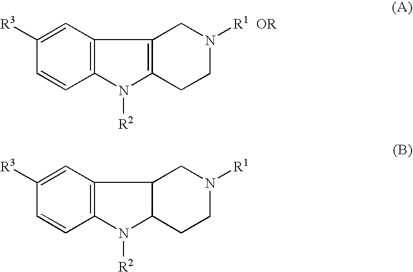
wherein:
R¹ is selected from a lower alkyl or aralkyl
R² is selected from a hydrogen, aralkyl or substituted heteroaralkyl
R³ is selected from hydrogen, lower alkyl or halo.

41. The method of claim 40, wherein aralkyl is PhCH₂— and substituted heteroaralkyl is 6-CH₃-3-Py-(CH₂)₂—.

42. The method of claim 40, wherein
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—.

43. The method of claim 37, wherein the hydrogenated pyrido (4,3-b) indole is selected from the group consisting of:
cis(±) 2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido[4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole;
2-methyl-8-bromo-2,3,4,5-tetrahydro-1H-pyrido[4,3-b]indole.

44. The method of claim 43, wherein the hydrogenated pyrido (4,3-b) indole is 2,8-dimethyl-5-(2-(6-methyl-3-pyridyl)ethyl)-2,3,4,5-tetrahydro-1H-pyrido[4,3-b]indole.

45. The method of claim 37 or 44, wherein the pharmaceutically acceptable salt is a pharmaceutically acceptable acid salt.

46. The method of claim 45, wherein the pharmaceutically acceptable salt is a hydrochloride acid salt.

47. The method of claim 37, wherein the hydrogenated pyrido (4,3-b) indole is 2,8-dimethyl-5-(2-(6-methyl-3-pyridyl)ethyl)-2,3,4,5-tetrahydro-1H-pyrido[4,3-b]indole dihydrochloride.

48. The method of claim 42, wherein R¹ is CH₃—, R² is H and R³ is CH₃—.

49. The method of claim 42 wherein R¹ CH₃CH₂— or PhCH₂—, R² is H—, and R³ is CH₃—.

50. The method of claim 42, wherein R¹ is CH₃—, R² is PhCH₂—, and R³ is CH₃—.

51. The method of claim 42, wherein R¹ is CH₃—, R² is 6-CH₃-3-Py-(CH₂)₂—, and R³ is H—.

52. The method of claim 42, where R² is 6-CH₃-3-Py-(CH₂)₂—.

53. The method of claim 42, wherein R¹ is CH₃—, R² is H—, and R³is H— or CH3—.

54. The method of claim 42, where R¹ is CH₃—, R² is H—, and R³ is Br—.

55. A method of slowing the onset of an age associated vision disturbance in a mammal, the method comprising administering to a mammal an amount of a hydrogenated pyrido (4,3-b) indole or pharmaceutically acceptable salt thereof effective to slow the onset of an age associated vision disturbance.

56. The method of claim 55, wherein the age associated vision disturbance is age associated cataracts.

57. The method of claim 56, wherein the hydrogenated pyrido (4,3-b) indole is a tetrahydro pyrido (4,3-b) indole.

58. The method of claim 56, wherein the hydrogenated pyrido (4,3-b) indole is a hexahydro pyrido (4,3-b) indole.

59. The method of claim 56, wherein the hydrogenated pyrido (4,3-b) indole is of the formula: 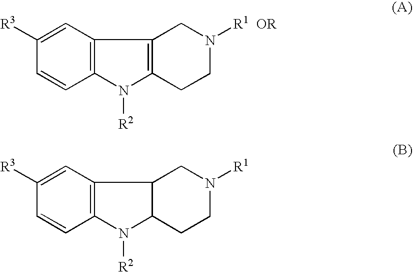
wherein:
R¹ is selected from a lower alkyl or aralkyl
R² is selected from a hydrogen, aralkyl or substituted heteroaralkyl
R³ is selected from hydrogen, lower alkyl or halo.

60. The method of claim 59, wherein aralkyl is PhCH₂- and substituted heteroaralkyl is 6-CH₃-3-Py-(CH₂)₂—.

61. The method of claim 59, wherein
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—.

62. The method of claim 56, wherein the hydrogenated pyrido (4,3-b) indole is selected from the group consisting of:
cis(±) 2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido[4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole;
2-methyl-8-bromo-2,3,4,5-tetrahydro-1H-pyrido[4,3-b]indole.

63. The method of claim 62, wherein the hydrogenated pyrido (4,3-b) indole is 2,8-dimethyl-5-(2-(6-methyl-3-pyridyl)ethyl)-2,3,4,5-tetrahydro-1H-pyrido[4,3-b]indole.

64. The method of claim 56 or 63, wherein the pharmaceutically acceptable salt is a pharmaceutically acceptable acid salt.

65. The method of claim 64, wherein the pharmaceutically acceptable salt is a hydrochloride acid salt.

66. The method of claim 56, wherein the hydrogenated pyrido (4,3-b) indole is 2,8-dimethyl-5-(2-(6-methyl-3-pyridyl)ethyl)-2,3,4,5-tetrahydro-1H-pyrido[4,3-b]indole dihydrochloride.

67. The method of claim 61, wherein R¹ is CH₃—, R² is H and R³ is CH₃—.

68. The method of claim 61, wherein R¹ CH₃CH₂— or PhCH₂—, R² is H—, and R³ is CH₃—.

69. The method of claim 61, wherein R¹ is CH₃—, R² is PhCH₂—, and R³ is CH₃—.

70. The method of claim 61, wherein R¹ is CH₃—, R² is 6-CH₃-3-Py-(CH₂)₂—, and R³ is H—.

71. The method of claim 61, where R² is 6-CH₃-3-Py-(CH₂)₂—.

72. The method of claim 61, wherein R¹ is CH₃—, R²is H—, and R³ is H— or CH₃—.

73. The method of claim 61, where R¹ is CH₃—, R² is H—, and R³ is Br—.

74. The method of claim 1, 19, 26, 29, 37, 44, 47, 55, 56, 63 or 66 wherein the mammal is a human.

75. The method of claim 74, wherein the human is elderly.

76. The method of claim 1, 19, 26, 29, 37, 44, 47, 55, 56, 63 or 66 wherein the method comprises administering a daily dose of the hydrogenated pyrido (4,3-b) indole to the mammal.

77. A method of improving the quality of life of a mammal, the method comprising administering to a mammal an amount of a hydrogenated pyrido (4,3-b) indole or pharmaceutically acceptable salt thereof effective to improve the quality of life of the mammal.

78-94. (canceled)

95. A method of prolonging the lifespan of a mammal, the method comprising administering to a mammal an amount of a hydrogenated pyrido (4,3-b) indole or pharmaceutically acceptable salt thereof effective to prolong the lifespan of the mammal.

96-112. (canceled)

113. A method of extending the lifespan of a cell in a mammal, the method comprising administering to a mammal an amount of a hydrogenated pyrido (4,3-b) indole or pharmaceutically acceptable salt thereof effective to extending the lifespan of a cell in the mammal.

114. The method of claim 113 , wherein the hydrogenated pyrido (4,3-b) indole is a tetrahydro pyrido (4,3-b) indole.

115. The method o f claim 113, wherein the hydrogenated pyrido (4,3-b) indole is a hexahydro pyrido (4,3-b) indole.

116. The method of claim 113, wherein the hydrogenated pyrido (4,3-b) indole is of the formula: 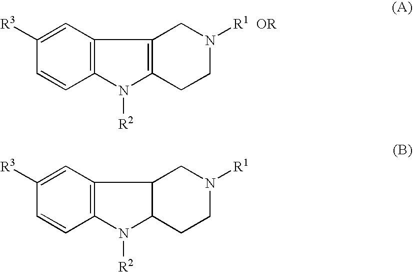
wherein:
R¹ is selected from a lower alkyl or aralkyl
R² is selected from a hydrogen, aralkyl or substituted heteroaralkyl
R³ is selected from hydrogen, lower alkyl or halo.

117. The method of claim 116, wherein aralkyl is PhCH₂— and substituted heteroaralkyl is 6-CH₃-3-Py-(CH₂)₂—.

118. The method of claim 116, wherein
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—.

119. The method of claim 113, wherein the hydrogenated pyrido (4,3-b) indole is selected from the group consisting of:
cis(±) 2,8-dimethyl-2,3,4,4a,5,9b-hexahydro-1H-pyrido[4,3-b]indole;
2-ethyl-2,3,4,5-tetrahydro-1H-pyrido[4,3-b]indole;
2-benzyl-2,3,4,5-tetrahydro-1H-pyrido[4,3-b]indole;
2,8-dimethyl-5-benzyl-2,3,4,5-tetrahydro-1H-pyrido[4,3-b]indole;
2-methyl-5-(2-methyl-3-pyridyl)ethyl-2,3,4,5-tetrahydro-1H-pyrido[4,3-b]indole;
2,8-dimethyl-5-(2-(6-methyl-3-pyridyl)ethyl)-2,3,4,5-tetrahydro-1H-pyrido[4,3-b]indole;
2-methyl-2,3,4,5-tetrahydro-1H-pyrido[4,3-b]indole;
2,8-dimethyl-2,3,4,5-tetrahydro-1H-pyrido[4,3-b]indole;
2-methyl-8-bromo-2,3,4,5-tetrahydro-1H-pyrido[4,3-b]indole.

120. The method of claim 119, wherein the hydrogenated pyrido (4,3-b) indole is 2,8-dimethyl-5-(2-(6-methyl-3-pyridyl)ethyl)-2,3,4,5-tetrahydro-1H-pyrido[4,3-b]indole.

121. (canceled)

122. The method of claim 121, wherein the pharmaceutically acceptable salt is a hydrochloride acid salt.

123. The method of claim 113, wherein the hydrogenated pyrido (4,3-b) indole is 2,8-dimethyl-5-(2-(6-methyl-3-pyridyl)ethyl)-2,3,4,5-tetrahydro-1H-pyrido[4,3-b]indole dihydrochloride.

124. The method of claim 118, wherein R¹ is CH₃—, R² is H and R³ is CH₃—.

125. The method of claim 118 wherein R¹ CH₃CH₂— or PhCH₂—, R² is H—, and R³ is CH₃—.

126. The method of claim 118, wherein R¹ is CH₃—, R² is PhCH₂—, and R³ is CH₃—.

127. The method of claim 118, wherein R¹ is CH₃—, R² is 6-CH₃-3-Py-(CH₂)₂—, and R³ is H—.

128. The method of claim 118, where R² is 6-CH₃-3-Py-(CH₂)₂—.

129. The method of claim 118, wherein R¹ is CH₃—, R² is H—, and R³ is H— or CH₃—.

130. The method of claim 118, where R¹ is CH₃—, R² is H—, and R³ is Br—.

131. The method of claim 22, 40, 59, or 116 wherein the hydrogenated pyrido (4,3-b) indole is of the formula:

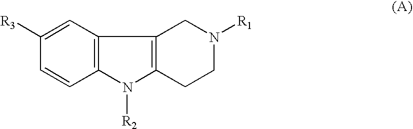

132. The method of claim 22, 40, 59, or 116 wherein the hydrogenated pyrido (4,3-b) indole is of the formula:

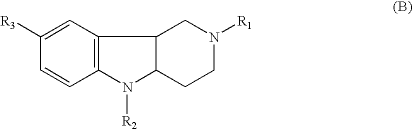

133. A sustained release formulation comprising a compound of the formula: 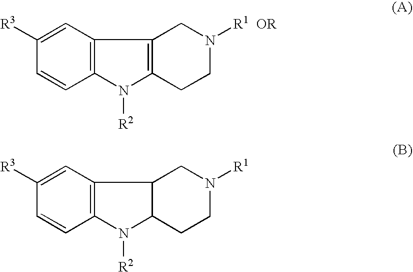
wherein:
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—,
or a pharmaceutically acceptable salt thereof.

134. A sustained release device comprising a compound of the formula: 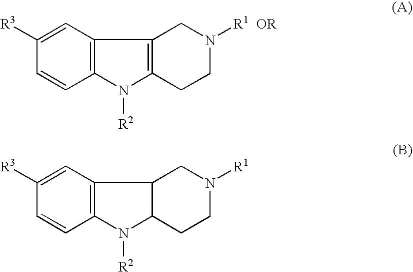
wherein:
R¹ is selected from CH₃—, CH₃CH₂—, or PhCH₂—
R² is selected from H—, PhCH₂—, or 6-CH₃-3-Py-(CH₂)₂—
R³ is selected from H—, CH₃— or Br—,
or a pharmaceutically acceptable salt thereof.

135. The sustained release formulation of claim 133, wherein the compound is 2,8-dimethyl-5-(2-(6-methyl-3-pyridyl)ethyl)-2,3,4,5-tetrahydro-1H-pyrido[4,3-b]indole or a pharmaceutically acceptable salt thereof.

136. The sustained release device of claim 134, wherein the compound is 2,8-dimethyl-5-(2-(6-methyl-3-pyridyl)ethyl)-2,3,4,5-tetrahydro-1H-pyrido[4,3-b]indole or a pharmaceutically acceptable salt thereof.

137. A kit comprising 2,8-dimethyl-5-(2-(6-methyl-3pyridyl)ethyl)-2,3,4,5-tetrahydro-1H-pyrido[4,3-b]indole or a pharmaceutically acceptable salt thereof and instructions for use.
